(19) United States
(12) Patent Application Publication
(10) Pub. No.: US 2004/0151717 A1
(43) Pub. Date: Aug. 5, 2004

(54) Use of polyclonal immunoglobulins

(76) Inventors: Hans Loibner, Vienna (AT); Gottfried Himmler, Vienna (AT)

Correspondence Address: BIRCH STEWART KOLASCH & BIRCH, PO BOX 747, FALLS CHURCH, VA, 22040-0747, US

(21) Appl. No.: 10/478,942

(22) PCT Filed: Mar. 19, 2002
(86) PCT No.: PCT/AT02/00088

(30) Foreign Application Priority Data
Jun. 1, 2001 (AT) A860/2001

(51) Int. Cl. A61K 39/395

(52) U.S. Cl. 424/130.1

(57) ABSTRACT

The invention relates to the use of a polyclonal immunoglobulin preparation for producing a vaccine formulation, containing antibodies of various specific natures for immunising individuals of the same species from which the immunoglobulins are supplied. Said actively immunising preparation can be used, for example, for the prophylaxis or treatment of cancer, auto-immune diseases, allergies and susceptibility to viral, bacterial or fungal infections.

The present invention relates to the use of polyclonal immunoglobulin preparations.

Higher organims are characterised by an immune system which protects them from potentially dangerous substances or microorganism. If a substance (antigen) enters the body, it is recognized as “foreign” and eliminated with the help of the immune system. Also “dengenerate” endogenous cells commonly are recognized by the immune system and eliminated.

The adaptive immune system of humans consists of two essential components, the humoral and the cellular immunity. The adaptive immune response is based on the clonal selection of B- and T-lymphocytes and in principle allows for the recognition of any antigen as well as for the build-up of an immunological memory. These characteristics of the adaptive immune system generally are usefully addressed in case of vaccinations.

Each B-cell produces an antibody of a certain binding specificity. This antibody is also present as a specific receptor in the membrane of the B-cell producing it. The humoral immune response against antigens recognized as foreign is based on the selective activation of those B-cells which produce such antibodies, which can bind to epitopes of the respective antigen. DNA-rearrangements in the course of B-cell differentiation play a decisive role for the large variety of antibodies.

In human serum, there are large amounts of antibodies of the most varying specificities, isotypes and subclasses. The total concentration of all immunoglobulins in the serum is 15-20 mg/ml; this means that about 100 g of immunoglobulins of the most varying specificities continuously circulate in blood. It is not possible to indicate the precise number of all antibodies with different specificity, the repertory of different B-cell clones in one human being is about 10⁹. In general, a certain antibody can bind various similar antigens, even though with different affinity and avidity.

With the help of endogenous regulating mechanisms, the immune system must maintain a homeostasis as regards the distribution and importance of these different specificities. One essential mechanism for this is the “idiotypic network” (Ann. Immunol. 125C: 373-89 (1974)). Against each idiotype of an antibody which determines the binding specificity of the latter, there exist anti-idiotypic antibodies which therefore bind to the idiotype of the first antibody as in an antigen recognition. According to this explanation model, the interactions between the idiotype-specific receptors on lymphocytes are responsible for the regulation of the immune system. These interactions apparently do in fact occur, since it has been shown that in the course of an immune response, also anti-idiotypic antibodies form against the antibodies primary-induced by the immune response. Since there exist anti-idiotypic antibodies against any antibody, lymphocytes basically are not tolerant relative to idiotypes of antibodies (William E. Paul, Eds., Fundamental Immunology, 3^{rd} Ed., Raven Press Ltd. New York, 1993, pp. 887-902).

Thus, the immune system consists of lymphocyte clones which are stimulated, or regulated, respectively, via immunoglobulins produced by other clones within the network. By the term Connectivity, the degree of cross-linking of the immune system is to be understood. An immune system with little connectivity contains a relative large amount of immune cell clones which are not influenced by idiotypic/anti-idiotypic interactions. By this type of interactions, not only the direct interactions are to be understood, i.e. those between two antibody binding sites. Primarily those actions which form indirectly, by a series of interactions of antibodies, are to be understood. Not only the B-cells and antibodies, but also T-cells with their receptors are involved (Immunol. Rev. (1988) 101:191-215).

The entire immune network has a certain “inner structure” within which the B-lymphocytes fall into various categories, i.e. produce immunoglobulins with different basic properties (Immunol. Rev. (1989) 110:37-61):

antibodies which exhibit an affinity to almost all other immunoglobulins (i.e. also with auto-affinity, i.e. they also react with themselves, “sticky antibodies”)

mirror-antibodies are antibodies which have an affinity to the idiotype of a certain antibody,

antibodies which have little affinity to “sticky antibodies”.

The higher the connectivity of a network, the larger the variety of cross-linking immunoglobulins. All these antibodies are in a certain balance. A shifting of the balance can cause pathologic phenomena (autoimmune diseases, allergy, cancer, susceptibility to infectious diseases). In this context, the occurrence of pathological phenomena is partly to be understood as an unregulated expansion of autoreactive clones which is caused by a defective or reduced connectivity.

Data show that the positive effect of the therapeutic application of high doses of polyclonal immunoglobulin (IVIG) in autoimmune diseases is to be attributed to a restoration of or an improvement in the connectivity of the immune system (Immunological Reviews (1989) 110:135-149; Scand. J. Immunol. (2000) 51:408-414; Immunology Today (1994) 15:341-342; Dermatol. clin. (2000) 18:447-457).

In the U.S. Pat. Nos. 5,562,902 and 5,965,130, the application of IVIG is described for cancer therapy.

One of the earliest applications of pooled immunoglobulin is the avoidance of infectious diseases. The immunoglobulin used in this way can lead to an anaphylactic shock in case of intravenous administration, and therefore it is administered i.m. in limited amounts (a few milliliters, corresponding to a few hundred milligrams of immunoglobulin).

It has also been observed that the prophylactic administration of serum immunoglobulins for preventing hepatitis has also lowered the incidence of various skin diseases in the treated population (Int. J. Dermatol. (2000) 39:628-631).

Various theories have been set up on account of various experimental observations on the mode of action of pooled serum globulin):

Influencing the phagocytosis via the Fc-Fc-receptor interaction (Ann. Intern. Med. (1991) 115:294-307);

Regulating the idiotypic/anti-idiotypic network; suppressing certain idiotypes (Int. J. Artif. Organs (1993) 16, Suppl.5: 189-195)

Suppressing the antibody production (Clin. Exp. Immunol. (1986)65:409-415)

Immunoglobulin-cytokine interactions (Immunol. Rev. (1994) 139:5-19)

Neutralisation of super antigens and toxins (New Engl. J. Med. (1991) 324:1633-1639)

Influencing of the complement system (J. Neuroimmunol. (1996) 71:227-229)

Acceleration of the immunoglobulin metabolism (Immunol. Today (1997) 18:592-598)

Neutrallisation of bacteria, fungi and viruses (Goodman and Goldman's The Pharmacological Basis of Therapeutics, McGraw-Hill, New York (1996):1291-1308).

Even though the mechanism of such immunoglobulin administrations cannot always clearly be attributed to one effect only, it can be observed that all appliers use a large amount of immunoglobulins for the therapy and assume a direct effect of the administered immunoglobulins. The amounts which commonly are used are a few hundred milligrams up to a few grams of immunoglobulin per kg of body weight per day, treatment mostly being carried out in more than one day (Dermatol. Clin. (2000) 18:447-457).

Pooled, polyclonal immunoglobulin (such as, e.g., intravenous immunoglobulin, IVIG or immune serum globulin) is prepared by fractionating pooled sera derived from thousands of donors. The hitherto used high doses for the therapy of many immunologically caused diseases has regularly led to a bottleneck in the supply with such preparations.

In WO 92/15885 A1, the production of a preparation containing monoclonal anti-idiotypic antibodies for the treatment of HIV infections is described. According to this document, suitable monoclonal antibodies were selected on the basis of their defined specificity to polyclonal antibodies and formulated into a vaccine. Polyclonal antibodies themselves are not used in the described preparation. Neither is the substitution of monoclonal antibodies by polyclonal antibodies suggested in this document for the person skilled in the art.

According to WO 91/114651 A1, anti-idiotypic antibody is used as an immunogenic mimic of a certain antigen. Here, both monoclonal and polyclonal antibodies are described, yet WO 91/114651 A1 discloses only the use of specific anti-idiotypic antibodies for the preparation of a vaccine, and not of polyclonal antibodies of different specificities.

Therefore, it has been the object of the present invention to provide a new and efficient possible therapy for such diseases, by means of which also bottlenecks in the supply can be avoided.

According to the invention, this object is achieved in that polyclonal immunoglobulins are employed in a completely new manner, i.e. for an immunisation. Thus, according to the invention, only slight amounts (compared to passive immunisation or other fields of use of immunoglobulins, in particular IVIGs in the piror art) of such a material must be employed, i.e. the amounts which are used for other active immunisations.

By an immunisation, an immune response is triggered which restores the connectivity of the immune system. The “sticky antibodies” present in polyclonal immunoglobulins, and the antibodies which have a weak affinity to “sticky antibodies”, produce an immune response which represent an enhancement of these antibody types. Likewise, in an autologous application, by the immunisation, anti-idiotypic antibodies against the pathological autoantibodies present in the polyclonal immunoglobulin can be induced. This can lead to a decrease in the production of the pathological antibodies.

Likewise, the present invention relates to the use of a polyclonal immunoglobulin preparation for the production of a vaccine formulation containing antibodies of different specificities for the immunisation of individual of the same species from which the immunoglobulins have been derived. For instance, according to the invention human immunoglobulin preparations for the treatment of humans are provided, while e.g. bovine or porcine immunoglobulins are used for the treatment of cattle or pigs, respectively.

As the polyclonal antibodies, antibodies of different specificities are utilized, e.g. the plurality of specificities which are found in human serum, or in pooled immunoglobulin fractions of human blood plasma, respectively.

According to the prior art, polyspecific antibodies are administered as immunoglobulin preparations, wherein an immunogenicity had neither been desired nor found. It has been surprising that polyspecific antibodies which are formulated to an inventive vaccine exhibited an advantageous effect as immunogens for the activation of the immune response. Despite non-specific immunogens, the reactivity relative to certain undesired antigens provably could be increased without undesired side effects. This reactivity is mainly effective against the allogenic antigens, such as tumor antigens or auto-antibodies which as such are not recognized as foreign by the body.

In a vaccine formulation according to the invention which contains human polyspecific antibodies, commonly approximately 5 μg-10 mg of immunoglobulins are provided in a volume of from 0.01 to 1 ml so as to treat patients. The preferred immunoglobulin amount is approximately 10 to 1000 μg, most preferred 50 to 750 μg.

The vaccine is suitable both for the prophylaxis and also for the therapy of diseases, wherein the primary syndromes are connected with tumor, infectious and autoimmune diseases.

The present invention avoids, or reduces, respectively, the above-mentioned disadvantages of the prior art, in that the immunoglobulin preparations are used in small amounts and such that they induce an immune response in the receiving organism. In this connection, it was surprising that the immunisation with polyspecific immunoglobulins could be achieved without undesired side effects, particularly since despite an unspecific activation of the immune response, the specific binding activity of tumor cells was significantly increased.

According to the present invention, a polyclonal immunoglobulin pool (e.g. IVIG or other gamma globulin formulations) can be used for an active immunisation.

In doing so, the immunoglobulin preparation is administered to the receiving organism in an amount typical of an active immunisation. The route of administration also corresponds to that common for active immunisations (e.g., subcutaneous, intradermal or intramuscular), preferred are the subcutaneous and intradermal modes of administration.

Preferably, an autologous immunoglobulin preparation is used as the polyclonal immunoglobulin preparation for the active immunisation of the same individual from which the preparation has been derived. The autologous administration of a polyclonal immunoglobulin preparation, i.e. the administration of the immunoglobulin preparation to the individual from whose immunoglobulin-containing body fluid the polyclonal immunoglobulin has been derived, therefore is a particularly advantageous embodiment of the present invention. An additional advantage of this autologous embodiment of the present invention is the fact that an infection of the immunoglobulin preparation from other individuals (e.g. viruses, such as hepatitis C or HIV), as may be present in pooled preparations, can be excluded. By individuals according to the present invention individual human or animal organisms are to be understood who have body fluids or tissues which contain antibodies. Of course, preferably the inventive preparation is used in vertebrates, particularly preferred in mammals, in particular in humans.

Preferably, one or more adjuvants are admixed to the polyclonal immunoglobulin preparation. By adjuvants, substances are to be understood which are capable of qualitatively and/or quantitatively improving the immune response for a given immunogen. The polyclonal immunoglobulin according to the present invention is administered in a form which allows for the triggering of an immune response. To enhance this immune response, therefore, the immunoglobulin preparation can be administered with adjuvants as are common in immunology.

As examples of adjuvants, the following shall be mentioned, without being restricted thereto: aluminum-containing adjuvants, in particular aluminum hydroxide, derivatives of lipopolysaccharide, Bacillus Calmette Guerin (BCG), saponins and derivatives thereof (e.g. QS-21), liposome preparations. Accordingly, in a preferred embodiment of the present invention, the working up of the antibody preparations as a vaccine formulation includes the addition of a substance selected from the group of adjuvants, in particular aluminum-containing adjuvants, lipopolysaccharide derivatives, Bacillus Calmette Guerin, liposomes or QS-21 (further preferred adjuvants have i.a. been described in Singh et al., Nat. Biotechnol. 17 (1999), pp. 1075-1081), immunostimulating cells, in particular dendritic cells, or other antigen-presenting cells, active agents, preferably cytokines, in particular granulocyte-macrophage-stimulating factor and/or the addition of formulating auxiliaries, in particular buffer substances, stabilizers or solubilizers, or mixtures of these substances.

In the patent specifications U.S. Pat. No. 5,965,130 as well as U.S. Pat. No. 5,562,902 and EP-0 750 514-A, lower amounts have been indicated (for a subcutaneous administration, 4 mg to 20 mg per kg of body weight per day), yet these amounts still are not in the range which is typically used for active immunisations. The typical amounts for active immunisations are approximately 100 nanograms per kg of body weight to 100 micrograms per kg of body weight per immunisation.

In the previously mentioned US patent specifications, one example is listed in which an amount of 200 micrograms of immunoglobulin, subcutaneously administered, shows an effect. This example is misleading insofar as there the immunoglobulin preparation had not been derived from the same species into which it was inocculated (human immunoglobulin was inocculated into a mouse), and the amount was used in mice and therefore again an amount of approximately 1 milligram per kg of body weight had been reached.

Preferably, of the polyclonal immunoglobulin preparation in the vaccine formulation, per immunisation, an amount of less than 200 micrograms per kilogram of body weight is used per immunisation, preferably less than 20 micrograms per kilogram of body weight, in particular less than 5 micrograms per kilogram of body weight. Yet, also immunoglobulin amounts of 200 nanograms up to 1 microgram per kilogram of body weight may suffice per immunisation. Commonly, the dose is standardised for one species and based on the mean body weight of the respective species.

The isolation of the immunoglobulins from human or animal body fluids that contain immunoglobulins (e.g. human serum or plasma) can be effected by sufficiently known methods. For this, precipitation methods, chromatographic (e.g. ion exchange chromatography, hydrophobic interaction chromatography or affinity chromatography with immunoglobulin-specific ligands, such as anti-IgG or anti-IgM or protein G and the like) or other methods or combinations of various methods may be employed. What is necessary for the inventive isolation of immunoglobulins from an individual is only that the desired immunogloublins are substantially separated from other undesired substances from the body. Preferred is a purity of 90%, particularly preferred is a purity of 98%; each based on the total protein content. It is also important for the present invention in this connection that the plurality of the immunoglobulins is largely maintained by the production method for the polyclonal immunoglobulin. In particular, preferably there shall not be any enrichment or depletion step (as is mentioned e.g. in J. Immunol. (1985) 135:1091-1096) for certain immunoglobulin specificities in the production method.

The immunoglobulin preparation as defined by the present invention is primarily composed of IgG, IgM and IgA, yet it is also possible to use only one specific class of immunoglobulins (e.g. only IgM or IgA or only IgG), or certain combinations of classes of immunoglobulins.

The term “immunoglobulin” or “antibody” as defined by the present invention therefore also comprises fragments or derivatives of the recovered antibody. As examples, the following shall be mentioned, without being restricted thereto: F(ab)2′ fragments, F(ab)′ fragments which may, e.g. be prepared by biochemical methods (e.g. by enzymatic cleavage) know per se. The term “derivative” in this connection comprises, e.g., antibody derivatives which can be prepared by chemical or biochemical methods known per se. In particular, the term also comprises products which can be prepared by chemical coupling of antibodies or antibody fragments with molecules that are capable of enhancing the immune response (such as, e.g. tetanus toxoid, Pseudomonas exotoxin, derivatives of lipid A, GM-CSF, IL-2, IL-12, C3d).

As the source of polyclonal immunoglobulin, pools of fluids containing immunoglobulins from different individual can be employed, yet also immunoglobulin-containing fluids of single individuals can be used.

Accordingly, a preferred embodiment of the present invention relates to the use of a polyclonal immunoglobulin preparation in native form, i.e. immunoglobulins that have been prepared without enrichment or depletion for certain immunoglobulin specificities, and which thus corresponds in its composition to a native immunoglobulin repertory present or circulating, respectively, in the respective organism, or in the respective body fluid (blood, lymph fluid, colostrum etc.), respectively.

Therefore, according to a further aspect, the present invention relates to the inventive use of immunoglobulins for preparing a vaccine formulation containing antibodies of various specificities, for the treatment of cancer, for the treatment of autoimmune diseases, for the prevention (the prophylaxis) or treatment of allergies and for the treatment of the susceptibility to viral, bacterial or fungus infections; each by immunisation.

The present invention also relates to a method for the prophylaxis or for the treatment of individuals in which a formulation prepared according to the invention is administered in an efficient amount, preferably a few micrograms to one hundred micrograms per kilogram of body weight, to the individual from whom the body fluid has been taken. This treatment method is primarily usable in case of autoimmune diseases, such as, e.g., systemic Lupus erythematosus, autoimmune thyroiditis, systemic vasculitis, Guillain-Barre Syndrome and anti-factor VIII:C autoimmune disease, in case of allergies or in case of cancer.

The treatment of healthy individuals may be carried out according to the invention as a prophylaxis against infections (such as, e.g., various skin diseases).

In general, the treatment can be carried out for therapeutic as well as prophylactic purposes.

In a further aspect, the present invention also relates to the use of an autologous immunoglobulin preparation for the production of an agent for immunomodulation.

In a further aspect, the present invention also relates to a vaccine containing human, polyspecific anitbodies to be administered to humans, obtainable by formulating a polyclonal, poly-specific human immunoglobulin preparation into a vaccine formulation. Preferably, this inventive vaccine is obtainable by formulating an immunoglobulin pool. Preferably, this vaccine also further comprises one or more adjuvants. According to a particularly preferred embodiment, the inventive vaccine contains immunoglobulins in an amount ranging from 5 μg to 10 mg.

The invention will be explained in more detail by way of the following examples to which, however, it shall not be restricted.

### EXAMPLE 1

### Production of an Immunoglobulin Preparation for Active Immunisation

0.83 ml of a suspension of Alu-gel (Alu-Gel S from Serva, 2% suspension; quality grade: adjuvant for the preparation of vaccines) is mixed under sterile conditions with 5 ml of a solution of rhesus monkey immunoglobulin (Sigma, USA, I4385, diluted to 2.5 mg/ml in 1 mM phosphate buffer, pH 6.0; 0.86% NaCl) and gently swivelled at room temperature for one hour. The suspension is sterile-filled in 0.5 ml aliquots into small puncture bottles.

### EXAMPLE 2

### Increasing the Anti-Tumor Cell Binding Activity of Immune Sera

Rhesus monkeys are immunised four times with 0.5 ml each of the vaccine prepared in Example 1 (day 0, 15, 29 and 60). The sera from the monkeys (pre- and immune sera) are tested in the cell ELISA for binding capacity to human tumor cells.

It is examined whether or not immunoglobulins which bind to human cancer cells can be detected in the monkey immune serum. For this purpose, the KATO III stomach cancer cell line is employed. These examinations are carried out by means of cell-ELISA tests as follows:

The wells of a microtiter plate are each incubated with 100 μl of a cell suspension of the cell line to be tested at the concentration of 2×10⁶ cells/ml in medium A over night at +4° C. After the supernatant has been sucked off, the plate is incubated with 50 μl of fixing solution per well for 5 minutes at room temperature. After the supernatant has been sucked off, 200 μl of blocking buffer B each are added by pipetting, and the plate is incubated for 1 hour at 37° C. After having been washed twice with 200 μl of washing buffer B each, 100 μl aliquots each of the monkey sera to be tested are incubated in dilutions of 1:10 to 1:100,000 in dilution buffer B for 1 hour at 37° C. After having washed the plate twice with 100 μl each of ice-cold washing buffer B, 100 μl of the peroxidase-conjugated anti-human-Ig-antibody (Zymed) each are added in a dilution of 1:1000 in diluting buffer A and incubated for 45 minutes at 37° C. The plate is washed three times with 100 μl each of ice-cold washing buffer B. The antibody binding is demonstrated by adding 100 μl each of the specific substrate, and the colour reaction is stopped after approximately 5 minutes by adding 50 μl each of stop solution. The evaluation is effected by measuring the optic density (OD) at 490 nm (wave length of the reference measurement is 620 nm).

Result (dilution 1:100 as shown in Table 1):

A clear increase in the binding capacity appears in the course of immunisation.

TABLE 1

| Sample | MOD |
|---|---|
| Pre-serum | 250 |
| Day 15 | 323 |
| Day 29 | 845 |
| Day 60 | 1224 |
| Washing buffer A: | 2% NaCl |
| | 0.2% Triton X-100 |
| | in PBS deficient |
| Washing buffer B: | 0.05% Tween 20 in PBS deficient |
| Blocking buffer A: | 5% fetal calf serum (heat-inactivated) |
| | in PBS deficient |
| Blocking buffer B: | 1% bovine serum albumin |
| | 0.1% NaN₃ |
| | in PBS deficient |
| Dilution buffer A: | 2% fetal calf serum (heat-inactivated) |
| | in PBS deficient |
| Dilution buffer B: | PBS-deficient |
| Staining buffer: | 24.3 mM of citric acid |
| | 51.4 mM of Na₂HPO₄ |
| | pH: 5.0 |
| Substrate: | 40 mg of o-phenylene diamine-dihydro- |
| | chloride |
| | 100 mg staining buffer |
| | 20 μl of H₂O₂ (30%) |
| Stop solution: | 4 N H₂SO₄ |

1. The use of a polyclonal immunoglobulin preparation for producing a vaccine formulation, containing antibodies of different specificities, for immunising individuals of the same species from which the immunoglobulins have been derived.

2. The use according to claim 1, characterised in that the polyclonal immunoglobulin preparation is an autologous immunoglobulin preparation for immunising the same individual from whom the formulation has been derived.

3. The use according to claim 1 or 2, characterised in that one or more immunologic adjuvants are admixed to the polyclonal immunoglobulin preparation.

4. The use according to any one of claims 1 to 3, characterised in that the polyclonal immunoglobulin preparation in the formulation is provided in an amount of less than 200 micrograms per kilogram body weight per immunisation, preferably of less than 20 micrograms per kg of body weight per immunisation, in particular of less than 5 micrograms per kg body weight per immunisation.

5. The use according to any one of claims 1 to 4, characterised in that the polyclonal immunoglobulin preparation is provided in native form in the formulation.

6. The use according to any one of claims 1 to 5 for preparing a vaccine formulation containing antibodies of different specificities, for the prevention or treatment of cancer by immunisation.

7. The use according to any one of claims 1 to 5 for preparing a vaccine formulation containing antibodies of different specificities, for the prevention or treatment of autoimmune diseases by immunisation.

8. The use according to any one of claims 1 to 5 for preparing a vaccine formulation containing antibodies of different specificities, for the prevention or treatment of allergies by immunisation.

9. The use according to any one of claims 1 to 5 for preparing a vaccine formulation containing antibodies of different specificities, for the prevention or treatment of the susceptibility to viral, bacterial or fungus infections, by immunisation.

10. A vaccine, containing human, polyspecific antibodies, to be administered to humans, obtainable by formulating a polyclonal, polyspecific human immunoglobulin preparation into a vaccine formulation.

11. A vaccine according to claim 10, obtainable by formulating an immunoglobulin pool.

12. A vaccine according to claim 10 or 11, characterised in that one or more adjuvants are contained therein.

13. A vaccine according to any one of claims 10 to 12, characterised in that it contains immunoglobulins in an amount ranging from 5 μg to 10 μg.
